(19) United States
(12) Patent Application Publication
(10) Pub. No.: US 2023/0104450 A1
(43) Pub. Date: Apr. 6, 2023

(54) Continuous Monitoring To Detect Changes in a User's Mental State to Implement Stepped Care

(71) Applicant: Koa Health B.V., Barcelona (ES)

(72) Inventors: Roger Garriga Calleja, Barcelona (ES); Teodora Sandra Buda, Barcelona (ES); João Guerreiro, Lisbon (PT); Jesus Alberto Omaña Iglesias, Barcelona (ES); Aleksandar Matic, Lloret de Mar (ES)

(21) Appl. No.: 17/494,730

(22) Filed: Oct. 5, 2021

(51) Int. Cl. G16H 20/70; G16H 40/63; G16H 50/20

(52) U.S. Cl. CPC G16H 20/70; G16H 40/63; G16H 50/20

(57) ABSTRACT

A system for determining a mental health state of a user and for adjusting output content accordingly includes a monitoring unit that monitors parameters of the user and acquires corresponding data, an analysis unit that extracts features from data acquired by the monitoring unit, a classification unit that detects change in the mental health state of the user based on the features extracted by the analysis unit and classifies the change in mental state of the user, a control unit that adapts the content to be output to the user by the output unit based on the detected change in the mental health state of the user classified by the classification unit, and an output unit that outputs content to the user based on the detected change in the mental health state of the user. A method determines the mental health state of the user and adjusts output content accordingly.

### TECHNICAL FIELD

The present invention relates to a system and method for determining a mental health state of a user and for adjusting content output or presented to the user by the system. The interaction of the system with the user is adapted based on an automated diagnosis by the system regarding the mental state of the user to ensure exposure of the user to content optimized to treat the detected mental health state.

### BACKGROUND

As the recent COVID-19 pandemic has doubled the rates of common mental health disorders such as depression and anxiety, there is a large and growing unmet need to remedy undesired symptoms of mental health conditions in the population. It is estimated that around 1 in 5 (21%) adults experienced some form of depression in early 2021. This is an increase compared to a comparable period up to November 2020 (19%) and more than double that observed before the COVID-19 pandemic (10%).

This increase in adverse mental health conditions has put a strain on mental health care professionals such as therapists and psychologists, whose numbers have remained constant. In addition, contact restrictions due to the pandemic have often exacerbated mental health disorders and have also posed a hurdle to treatment, as patients could not easily meet mental health care professionals in person.

It is an object of the present invention to alleviate or completely eliminate the drawbacks associated with existing methods of delivering mental health therapies and treatments. In particular, it is an object of the present invention to ensure that all people receive adequate assistance with their mental health conditions without putting an undue strain on mental health care professionals.

### SUMMARY

A system or method according to the present disclosure enables individual users to receive tailored mental health care recommendations and resources, such as recommended reading, recommended apps focusing on mindfulness, healthy sleeping, etc., that are available to the users wherever they are in their clinical trajectory and as early as possible in that journey. A highly curated and personalized service of behavioral health and mental wellness enables the appropriate level of care for individuals to be identified with the ability seamlessly to move up or down a stepped care continuum of lower and higher intensity interventions to meet the users' evolving mental health needs.

The present disclosure relates to a system for determining a mental health state of a user and adjusting output content accordingly; the system comprising a monitoring unit, configured to monitor parameters of a user and acquire corresponding data; an analysis unit, configured to extract features from data acquired by the monitoring unit; a classification unit, configured to detect a change in a mental health state of the user based on the features extracted by the analysis unit and to classify the change in mental state of the user; a control unit configured to adapt or select the content to be output to the user by the output unit based on the detected change of the mental health state of the user classified by the classification unit, and an output unit, configured to output content to a user based on a detected mental health state of the user or a change in the mental health state of the user. Another aspect of the invention relates to a method for determining a mental health state of a person and adjusting output content accordingly.

Other embodiments and advantages are described in the detailed description below. This summary does not purport to define the invention. The invention is defined by the claims.

### BRIEF DESCRIPTION OF THE DRAWINGS

The accompanying drawings, where like numerals indicate like components, illustrate embodiments of the invention.

FIG. 1 is a schematic diagram of a stepped mental health care system that is part of a computing system that implements a smartphone app.

FIG. 2 shows a general concept of the operation of a system according to the present disclosure.

FIG. 3 is a flow chart demonstrating the creation of a system according to the present disclosure and the operation of such a system.

FIG. 4 illustrates an example of the operation of a system according to the present disclosure, the system being applied to an individual user.

FIG. 5 is a table of data collected for a user during week 0.

FIG. 6 is a table of data collected for the user during week 2.

FIG. 7 is a table of change-based features computed between week 4 and week 2.

### DETAILED DESCRIPTION

Reference will now be made in detail to some embodiments of the invention, examples of which are illustrated in the accompanying drawings.

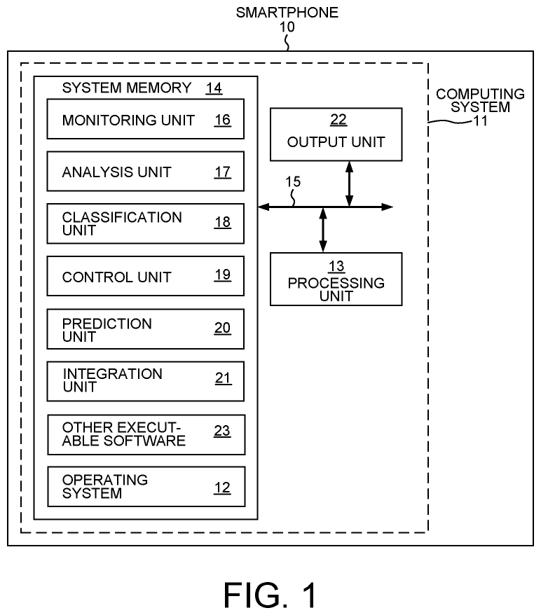

FIG. 1 is a schematic diagram of the components of an application program running on a smartphone 10, which is a mobile telecommunications device. The mobile application (app) forms part of a computing system 11. In one embodiment, the mobile app runs as modules of an application program on the computing system 11. In another embodiment, at least some of the functionality of the mobile app is implemented as part of the operating system 12 of smartphone 10. For example, the functionality can be integrated into the iOS mobile operating system or the Android mobile operating system. In yet another embodiment, at least some of the functionality is implemented on the computing system of a remote server that is accessed over the air interface from smartphone 10. The wireless communication modules of smartphone 10 have been omitted from this description for brevity.

Components of the computing system 11 include, but are not limited to, a processing unit 13, a system memory 14, and a system bus 15 that couples the various system components including the system memory 14 to the processing unit 13. Computing system 11 also includes machine-readable media used for storing computer readable instructions, data structures, other executable software and other data. Thus, portions of the computing system 11 are implemented as software executing as the mobile app. The mobile app executing on the computing system 11 implements a stepped mental health care system for determining a mental health state of a user and for adjusting output content accordingly.

The stepped mental health care system comprises various units of the computing system 11, including a monitoring unit 16, an analysis unit 17, a classification unit 18, a control unit 19, a prediction unit 20, an integration unit 21, and an output unit 22. The units of the stepped care system are computer readable instructions and data structures that are stored together with other executable software 23 in system memory 14 of the computing system 11.

The monitoring unit 16 is configured to monitor parameters of a user of the mobile app and to acquire corresponding data. The analysis unit 17 is configured to extract predetermined features from data acquired by the monitoring unit 16. The classification unit 18 is configured to detect a change in the mental health state of the user based on the predetermined features extracted by the analysis unit 17 and to classify the change in the mental state of the user. The control unit 19 is configured to adapt or select the content to be output to the user by the output unit 22 based on the detected change of the mental health state of the user classified by the classification unit 18. The output unit 22 is configured to output content to the user based on the detected mental health state of the user or based on a change in the mental health state of the user.

The stepped mental health care system can be employed in the context of e-health, where a user interacts with an electronic device or system in order to assess the user's mental state and to strive to improve that mental state.

Typically a user who is experiencing symptoms of depression turns to a clinician, who then assesses the severity of the depression using questionnaires and standard depression scoring methods. Based on this assessment, the clinician suggests a course of action, such as the use of a mindfulness app on the user's smart phone to aid the user to recognize positive aspects of life. Conventionally, the user would then regularly consult the clinician to assess whether any progress in the user's condition has been achieved and whether the course of action should be adjusted. The stepped mental health care system obviates the need for such routine follow-up consultations by automatically providing a diagnosis of the user's mental state, in particular any changes in the user's mental state, based on physiological data of the user. In addition, the stepped care system automatically adjusts the content output to the user according to the changes that are detected in the user's mental state. For example, if the system detects a change from mild depression to moderate depression, the system may change the delivered content from presenting the user with a mindfulness app to presenting the user with a sleep management and time management app and prompting the user to spend more time with friends or consult a self-help book.

The output unit 22 of the computing system 11 is configured to output content to the user based on the detected mental health state of the user. For example, the output unit 22 may be set up regularly to present the user with a program guiding the user through a breathing exercise. Over the course of two weeks of the user performing the breathing exercises, the monitoring unit 16, which is connected to one or more sensors, monitors physiological parameters of the user, such as stress level, heart rate, pupil dilation, skin conductivity, sleep patterns, and the number of steps taken per day. The analysis unit 17 then extracts predetermined features or feature vectors from the data regarding physiological parameters acquired by the monitoring unit 16 to detect changes in time of one or more features. For example, the analysis unit 17 may detect that due to breathing exercises, the user is more relaxed and thus shows an improved sleep pattern. The classification unit 18 is configured to classify the mental state of the user based on the changes detected by the analysis unit 17 and uses this improvement in relaxation and sleep pattern to classify the mental state of the user in the class “mild anxiety”, whereas previously the user showed moderate anxiety and had difficulty sleeping. The control unit 19 is configured to adapt the content output to the user by the output unit 22 based on the user's detected mental health state as classified by the classification unit 18. The control unit 19 then adjusts the content presented to the user based on the user's detected mental health state or based on the detected change in the user's mental health state, for example, the change from “moderate anxiety” to “mild anxiety”. For example, the new content presented to the user may be a mindfulness diary in which the user records positive aspects of daily life rather than a program for breathing exercises.

The stepped mental health care system automatically assesses the user's mental health state based on physiological data of the user, such as heart rate, pupil dilation, activity profile, sleep time, resting time, response time, and skin conductivity, and immediately adapts the content presented to the user to the detected health state in order to ensure that the individual user always receives content tailored to the user's specific needs. This ensures optimal care for the user while reducing the burden on mental health care professionals and reducing the need for face-to-face interactions.

The monitoring unit 16 is configured to acquire one or more of: physiological data of the user, data stemming from contextual input from the user such as ecological momentary assessment data, data from electronic health records, data reflecting the behavior of the user, or any other type of data directly or indirectly indicative of the user's mental health state. The monitoring unit 16 receives input from one or more sensors that can be different from each other and can be present on one device or multiple separate devices. The monitoring unit 16 can use sensors such as a digital camera, a microphone, a gyrometer, an accelerometer or any other sensor suitable for measuring a physiological state of the user or capturing a parameter indicative of the user's behavior or any other parameter to be monitored by the monitoring unit 16.

The control unit 19 is configured to adapt or select the content output to the user by the output unit 22 based on the user's detected mental health state or a detected change in the user's mental health state as classified by the classification unit 18. The type of content to be presented can be modified by the control unit 19. For example, the output unit 22 can be controlled to initially present a first type of content following a first intervention rationale, e.g., a mindfulness app, tailored to a first detected metal state of the user, and subsequently can be controlled to present a second type of content following a second intervention rationale, e.g., a program for breathing exercises, tailored to a second detected metal state of the user.

The prediction unit 20 is configured to predict a change in the user's mental health state, and thus also a change in the classification result of the classification unit 18, in the future based on the changes detected by the analysis unit 17. Thus, the stepped mental health care system leverages change-based features to predict a change in the user's mental state, in particular in the severity of symptoms of the user's mental condition, and therefore enables a stepped care approach in that content presented to the user matches the detected mental state or change in mental state. For example, the system may predict that the mental state of a user having a mental state classified as “mild anxiety” will in the near future, e.g., in two weeks, change to “moderate anxiety” because the analysis unit 17 has detected changes in the feature values extracted from the acquired physiological data that indicate reduced physical activity, reduced sleep and rest and reduced social interaction. The predicted change may be classified as a “step up” corresponding to an increase in symptoms and thus in required effectiveness of the content presented, “step down” corresponding to a decrease in symptoms and thus in a milder form of presented content being sufficient, or “neutral” if no change has been detected.

The prediction unit 20 and the classification unit 18 can be combined into a single module so that the classification unit 18 is not only configured to detect a current change in the mental state of the user but also is configured to predict a future change of the mental state of the user. In other words, the classification unit 18 can identify trends in the user's mental state and predict future changes in the user's mental state.

The analysis unit 17 detects changes in the features extracted from the data acquired by the monitoring unit 16 and changes in data output by the classification unit 18. The analysis unit 17 can be realized as a machine-learning based system, such as a neural network, in particular a convolutional neural network. In one embodiment, the analysis unit 17 is configured to determine changes over time in predetermined features extracted from data acquired by the monitoring unit 16 and to determine a change in the user's mental health state based on the changes in the predetermined features. The monitoring unit 16 is further configured to receive data reflecting the conscious mental state of the user, such as data obtained from clinical questionnaires.

In another embodiment, the stepped mental health care system also includes the integration unit 21, which is configured to integrate and synchronize data regarding the user's physiological parameters and data reflecting the user's conscious mental state to reflect the user's subconscious mental state. Thus, the system integrates data indicating how the user consciously feels (e.g., questionnaire data) with data indicating the user's physiological state that the user cannot influence. This increases the accuracy of determining the user's mental state. The data reflecting the conscious mental state of the user and the physiological data obtained from sensors, for example, present in the user's smart phone or a wearable such as a wrist band or smart watch, are integrated into a common data base or synchronized, for example by co-registering with a temporal stamp to link data obtained at the same time.

In addition, the classification unit 18 can include a neural network, and the data from the integration unit 21 may be used to train the neural network. In this case, data from clinical questionnaires, such as a defined depression level, are used as a target. Then physiological data of the user, data from electronic health records, data stemming from contextual input from the user, such as ecological momentary assessment data, and data reflecting the user's behavior are input into the classification unit 18.

The prediction unit 20 may be configured to predict future changes in the user's mental health state, such as an increase in the severity of anxiety symptoms, based on the changes detected by the classification unit 18, such as a reduction in sleep and activity, and the data acquired by the monitoring unit 16 reflecting the user's conscious mental state, such as the user reporting an increased feeling of gloom. Thus, the prediction unit 20 may not only leverage the changes in the feature vectors extracted from the physiological data, but may also draw on data reflecting the conscious mental state of the user.

The control unit 19 is configured to adapt the content output to the user by the output unit 22 based on a stepped care paradigm using the classification result of the classification unit 18 and/or the prediction result of the prediction unit 20. In the stepped care paradigm, defined classes of mental states are associated with defined recommended contents. For example, an increase in symptom severity or a worsening in the user's mental state is regarded as a “step up” in the stepped care paradigm, and a decrease in symptom severity or an improvement in the user's mental state is regarded as a “step down” in the stepped care paradigm.

If the control unit 19 is configured to adapt the content output to the user by the output unit 22 based on a stepped care paradigm using the prediction result of the prediction unit 20, such as an impending worsening of symptoms, the control unit 19 may preemptively adjust the content to avoid the worsening of symptoms. In other words, the content may be adjusted based on a predicted change in mental state prior to that change in mental state actually occurring.

The stepped mental health care system is configured automatically to determine the user's mental health state at a predetermined time-interval, such as daily, weekly or monthly, such that the length of the time-interval is determined by the control unit 19 based on the classification result of the classification unit 18 and/or on the prediction result of the prediction unit 20. For example, if a worsening of the user's mental state is detected or predicted, the system determines the mental state more frequently, such as daily, in order to ensure that no changes in the mental state are missed.

In one embodiment, the output unit 22, the monitoring unit 16, the analysis unit 17, the classification unit 18, the prediction unit 20 and/or the integration unit 21 are present in one single device, preferably smartphone 10. For example, the output unit 22 can control a graphical user interface, the monitoring unit 16 can be coupled to a camera of smartphone 10 that is used to capture a photopletysmogram (PPG) to detect the user's heart rate or to determine pupil dilation of the user. The monitoring unit 16 can also gather data from a gyroscope present on smartphone 10 or GPS data to determine the user's activity level. The analysis unit 17, the classification unit 18, the control unit 19, and/or the prediction unit 20 may all be realized as a central processing unit of the smartphone 10. The monitoring unit 16 can also include devices separate from the smartphone 10, such as fitness trackers, smart watches, smart wrist bands and other wearables.

The stepped mental health care system can also include an alert unit configured to output an alert via a device separate from the system, such as via a computer terminal present in a physician's office or a laptop, tablet or any other electronic device if the classification result generated by the classification unit 18 or the prediction result generated by the prediction unit 20 falls within a predefined class, such as a class associated with a mental condition that has a severity above a defined severity threshold. For example, if “severe depression” has been determined to be the user's mental state, an alert can be displayed on a physician's device, such as via a wireless connection or via a short message service (SMS).

The alert unit can also be configured to output an alert if there has been no detectable change in the classification result by the classification unit 18 or the prediction result by the prediction unit 20 for a predetermined time interval. For example, a physician may be alerted to the fact that an ongoing presentation of content to the user, such as a mindfulness app, may not have the desired effect.

In another embodiment, a novel method determines a mental health state of a user and adjusts output content accordingly. The method is implemented using the stepped mental health care system. The method involves monitoring parameters of the user and acquiring corresponding data using the monitoring unit 16. The analysis unit 17 is used to extract predetermined features from the data acquired by the monitoring unit 16. A change in the mental health state of the user is detected by the classification unit 18 based on the predetermined features. The classification unit 18 classifies the detected change in the mental state of the user into pre-defined classes. The control unit 19 is used to adapt or select the content to be presented to the user by the output unit 22 based on the detected change in the user's mental health state classified by the classification unit 18. The output unit 22 outputs content to the user based on the detected change of the mental health state of the user.

In the monitoring step, one or more of the following data are acquired: physiological data of the user, data stemming from contextual input from the user such as ecological momentary assessment data, data from electronic health records, data reflecting the behavior of the user, or any other type of data directly or indirectly indicative of a user's mental health state.

The method for determining a user's mental state and adjusting output content also includes using the classification unit 18 to determine changes in time in the predetermined features extracted from data acquired by the monitoring unit 16 and to determine any change in the user's mental health state based on the changes of the predetermined features. The method also includes the step of predicting a classification result of the classification unit 18 in the future based on the predetermined features extracted by the analysis unit 17 or based on changes in the predetermined features detected by the classification unit 18.

The method further includes the steps of acquiring data reflecting the user's conscious mental state and integrating data reflecting the user's conscious mental state with data regarding the user's physiological parameters that reflect the user's subconscious mental state. The monitoring unit 16 is used to acquire data reflecting the conscious mental state of the user, such as data stemming from clinical questionnaires, and physiological parameters of the user using one or more sensors. The integration unit 21 integrates and synchronizes the data regarding physiological parameters of the user that reflect the user's subconscious mental state with data reflecting the conscious mental state of the user. These steps more accurately determine the user's mental state because both conscious experience of the user as well as physiological data reflecting the subconscious state of the user are considered.

The prediction step involves predicting a future change in the user's mental health state (corresponding to a classification result of the classification unit 18) based on changes detected by the analysis unit 17 and the data reflecting the user's conscious mental state acquired by the monitoring unit 16. For example, the user can be prompted to provide information on how the user feels. In this manner, not only are changes detected in the feature vectors extracted by the analysis unit 17 considered, but also the user's conscious experience is taken into account.

The adapting step performed by the control unit 19 involves adapting the contents output to the user by the output unit 22 based on a stepped care paradigm using the classification result of the classification unit 18. Classes of mental states defined by the stepped care paradigm are associated with predetermined recommended contents. The control unit 19 selects the contents to be output to the user by the output unit 22 based on a stepped care paradigm using the classification results of the classification unit 18. In the stepped care paradigm, defined classes of mental states are associated with defined recommended contents.

The method is performed at predetermined time intervals, such as daily, weekly or monthly, to determine the mental state of the user. The length of the time interval is determined by the control unit 19 based on the classification results of the classification unit 18 or based on the prediction results of the prediction unit 20.

The method may further involve outputting an alert via a separate device if it is determined that the classification results of the classification unit 18 or the prediction results of prediction unit 20 fall within a predefined class, such as a class associated with a mental condition that has a severity above a predetermined severity threshold. The method may further involve outputting an alert via a separate device if there has been no detectable change in the classification results of the classification unit 18 or the prediction results of prediction unit 20 over a predetermined time interval.

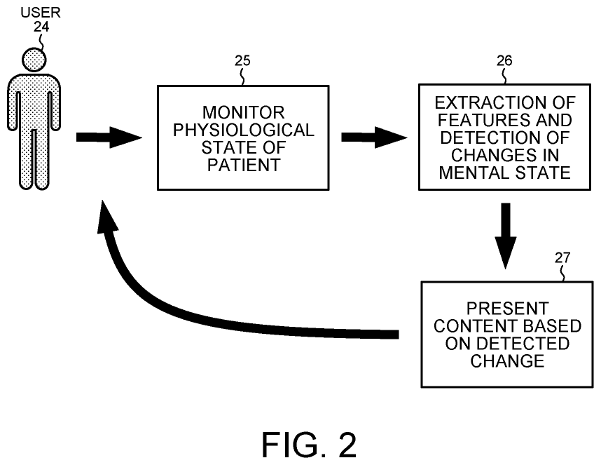

FIG. 2 is a flowchart of three general steps in the operation of the stepped mental health care system. In a first step 25, monitoring unit 16 is used to acquire data indicative of the physiological state of a user 24. A physiological parameter measured by the monitoring unit 16 may be blood pressure, pulse rate, skin conductivity, breathing rate, temperature, pupil dilation, gaze direction, or a physiological parameter reflecting the behavior of user 24, such as sleep pattern, activity profile, number of steps taken in a day, screen time, rest time, or time taken for social interaction or a movement profile. Any other suitable parameter reflecting the physiological state of user 24 may also be monitored.

The monitoring unit 16 acquires these data preferably passively, without any specific action of user 24 being required, and in some cases without user 24 even noticing. For example, the monitoring unit 16 may acquire activity data originally obtained from the user's smartphone 10 or measurements of the user's heart rate obtained from a smart watch or from the camera of the user's smartphone 10. The data acquired in the first step 25 is then stored in a database.

The method for providing stepped mental health care can also be used to diagnose many mental health disorders and illnesses, such as generalized anxiety disorder, panic disorders, phobias, obsessive-compulsive disorders (OCD), post-traumatic stress disorder (PTSD), attention deficit disorder (ADD), and attention deficit hyperactivity disorder (ADHD), depressive disorder, dysthymia, depression in the elderly, postpartum depression, stress or mild depression caused by co-morbidity with other health conditions such as strokes, cardiac procedures, cancer treatments, major accidents, major surgeries and cognitive impairments related to aging. All of these disorders and illnesses are described and defined in the Diagnostic and Statistical Manual of Mental Disorders, Fifth Edition (DSM-V).

In a second step 26, the analysis unit 17 extracts feature vectors from the acquired data and detects changes in the features relative to previous measurement or relative to an expected text book value. Based on this analysis, the mental state of the user is classified, for example by drawing on standard categories such as the classes of depression defined in the Diagnostic and Statistical Manual of Mental Disorders, Fifth Edition (DSM-V). The mental disorders are categorized as “mild”, “moderate”, “moderately severe”, and “severe”.

Based on the detected change and classification of the mental state of the user, in a third step 27, the control unit 19 adapts the contents presented to user 24 as indicated by the feedback loop in FIG. 2. For example, if a worsening in the user's mental state is detected, a more effective content will be presented to user 24. For example, instead of a mindfulness app, a guided self-help program for depression will be presented.

Thus, three steps are performed by the stepped mental health care system. The first monitoring step 25 involves capturing behavior, physiological data, and cognitive signals using sensors on smart devices. In the second step 26, changes are detected with regards to standard clinical measurements, such as from the Diagnostic and Statistical Manual of Mental Disorders (DSM-5), which is used by clinicians and researchers to diagnose and classify mental disorders based on change-based features. The third step 27 implements stepped care in which higher or lower intensity content is delivered to user 24 based on detected changes in the user's mental state.

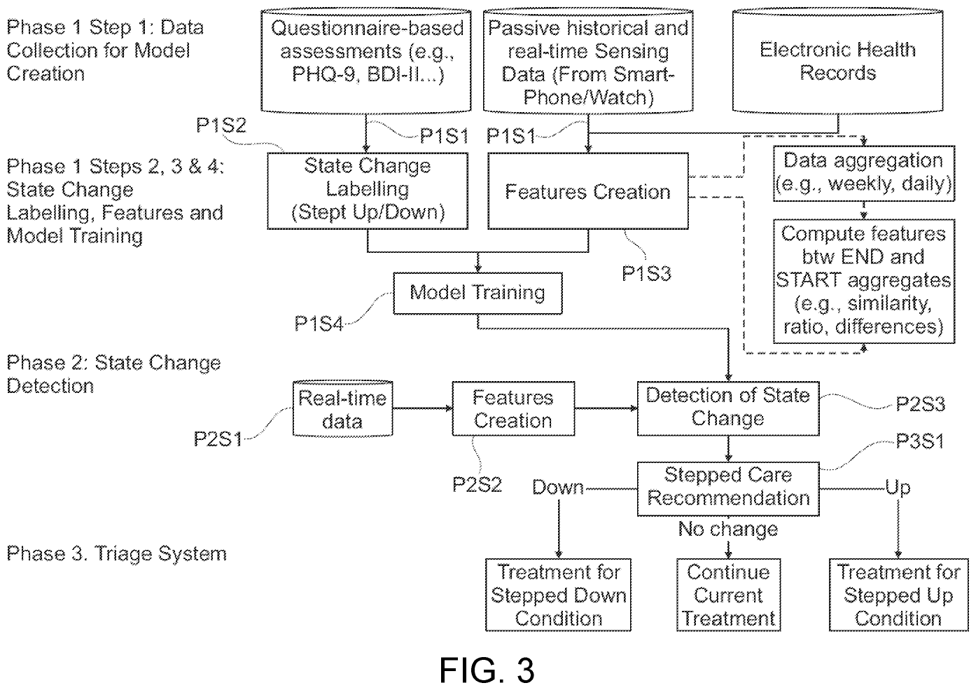

FIG. 3 is a flow chart showing the structure and operation of the novel stepped mental health care system. In FIG. 3, phase 1 shows the steps required for training a convolutional neural network (CNN) to be capable of detecting state changes in the feature vectors extracted from physiological real-time data acquired from an individual user of the system.

Phases 2 and 3 show the steps after the CNN has been trained in which the mental state of the individual user is diagnosed based on physiological real-time data acquired from the individual user and in which the content presented to the user is adapted according to a stepped care paradigm (also referred to as a triage system in FIG. 3).

Phase 1 includes four steps in which the CNN (also referred to as the model in FIG. 3) is trained. In the first step P1S1 of phase 1, data are collected for the creation of the CNN model. In step P1S1, the data from various sources, such as from smartphone sensors, wearables, electronic health records, questionnaire data, text notes from user diaries or clinician notes, or voice recordings, are collected and stored in a database.

The collected data may be obtained in part from the user's state assessment through questionnaires (e.g., the PHQ-9 BDI-II questionnaires for depression as outlined in the DSM-V) and in part from passive data measured by sensors, such as step count, sleep duration, heart rate, screen time, stationary time, etc. The results of the questionnaires are collected at least twice, separated by a period of time over which the system assesses any change in mental state. During a desired data collection period, the passive data is collected with a certain time granularity, e.g., daily.

In step P1S1, historical data concerning users are coupled with the ground-truth information regarding symptom severity. Historical data about users can include passive data collected from users' devices, such as smartphone sensors or smartwatches. This data can be used as a proxy or to represent day-to-day behavior (such as location, physical activity, sleep, light, social interactions) and physiological states (such as through ECG, heart rate, heart rate variability, electrodermal activity, screen swiping patterns, reaction times, etc). Ground truth information about symptom severity that is necessary for training the CNN can be collected through clinical assessments or from Electronic Health Records (EHR). In addition, ground truth information can be collected by questionnaire-based self-reports, such as Ecological Momentary Assessments (EMA) or standard scales, such as PHQ-9 (depression), wellbeing (WHO-5), or anxiety (GAD-7) that are known from the DSM-V.

Data is aggregated into various categories corresponding to different time intervals (hourly, daily, weekly) from these data sources. For instance, data obtained from smartphone sensors can be aggregated into the following features based on availability:
Daily step count (pedometer)
Daily activity summaries: percentage time stationary, walking, automotive, running; total minutes stationary, walking, automotive, running.
Daily screen time
Hourly activity counts
Hourly screen counts
Daily time sleeping
Swiping patterns

In the second step P1S2 of phase 1, state change labeling in performed. Questionnaire data reflecting the conscious mental state of an individual user is classified into severity level. For example, for PHQ-9 the result is assigned a severity level according to the score: 0-4=“none”, 5-9=“mild”, 10-14=“moderate”, 15-19=“moderately severe”, 20-27=“severe”. Then, the results from the same individual from two different points in time are compared, and the change over the time period between the two assessments is labeled as “step up”, “no change” or “step down”.

The change in mental state is labeled “step up” if the second result is in a greater severity level than the prior result. “No change” signifies that both results are within the same severity level. The change in mental state is designated “step down” if the second result is at a lesser severity level than the prior one.

To determine the change in mental state, in the second step P1S2 of phase 1, the data from questionnaires is used. In order to compute a change, the users must report their mental states (e.g., symptom severity) via the same assessment method, e.g., the same questionnaire, at least two times with a certain time separation between them (e.g., two weeks). For instance, for the questionnaire PHQ-8, the severity of depression symptoms is defined based on the value of the result with the following ranges : 0-4=“none”, 5-9=“mild”, 10-14=“moderate”, 15-24=“severe”. The state change is labeled as “step up” if the user increased the severity range within a certain time period (i.e., between one and the subsequent measurement). Conversely, “step down” reflects the situation in which the symptoms became less severe over a certain time period, i.e., the symptom severity range is lower after a certain time period (i.e., from one to the subsequent measurement). “No change” indicates that the user remained in the same symptom severity range.

In the third step P1S3 of phase 1, features and/or feature vectors are created from the collected data. The collected data is transformed into features and/or feature vectors that capture the differences between the physiological data reflecting the user's behavior between two different moments in time. In that sense, the data is aggregated at a certain time interval level (e.g., daily level or weekly level) over the period of time that the system is meant to detect the state change. For that purpose, a set of functions that combine the aggregated data at the end of the time period and the aggregated data at the beginning of the time period is applied, resulting in the features for the detection model. These functions include similarity measures, ratios and differences between the aggregated data at the start and at the end of the period.

In this example, the feature creation process is divided into two stages. In the first stage, the passive data is aggregated in order to capture the physiological data reflecting behavior or cognitive states at the beginning and at the end of the evaluated period. In that sense, a certain time period around the starting point and the end point is defined, such as the week before the first questionnaire measurement and the week before the second questionnaire. Then, the collected data from passive sensors are aggregated over those points in time by computing a set of variables, such as:
Average across days
Total sum
Standard deviation across days
Variance across days
Minimum value
Maximum value
Highest N (e.g., 3) values
Lowest N (e.g., 3) values
Average swiping speed
Total distance covered in swiping
Inefficiency

In the second stage of feature creation, the set of variables is transformed into change-based features that are used in the following step for modeling. These features are designed to reflect behavior and cognitive state changes between the start of the period and the end of the period. Similarity measure features are then created.

For example, for each of the highest N and lowest N types of variables (e.g., number of steps taken), a similarity measure feature is computed. This similarity measure is, for instance, the cosine similarity between the N highest/lowest values at the start and the N highest/lowest values at the end of the period. The N values in both cases are ordered by the value. For example, if N=3 (i.e., the lowest three or the highest three values are selected from a given period, in this example one week), the values in the first week are: Mon=3 (corresponding to three steps being taken that day), Tue=4, Wed=2, Thu=7, Fri=1, Sat=6, Sun=5, and the values in the second week are: Mon=2, Tue=6, Wed=3, Thu=4, Fri=0.5, Sat=1.5, Sun=7. Then, the similarity measure on the lowest N=3 is computed between the vector for the first week: (1,2,3) corresponding to the three lowest values in the first week and the vector for the second week: (0.5,1.5,2) corresponding to the three lowest values in the second week.

Regression slope features are also generated. The daily values of each data type are used to compute the regression slopes. For each data type, a linear regression is fitted using all the daily values available by using the day position within the period from the start to the end as the independent variable and the value as the dependent variable. The slope of the fitted regression is saved as a feature.

Ratio features are also generated. For each of the aggregated variables (e.g., averages, total sum, standard deviation, total distance covered swiping), the ratio between the value at the end of the period and the value at the beginning of the period is computed and saved as a feature.

Difference features are also generated. For each of the aggregated variables (e.g., averages, total sum, standard deviation, total distance covered swiping), the difference between the value at the end of the period and the value at the beginning of the period is computed and saved as a feature.

In the fourth step P1S4 of phase 1, the CNN model is trained using the data obtained in the first three steps P1S1-P1S3. In the fourth step P1S4, the features created in prior steps are used to train a state change model. There are two variants of how the fourth step P1S4 is performed. In the first variant, a model for step up and a separate model for step down are built. In the second variant, a single multiclass model to detect step up, down or no change is built. Thus, a machine learning model (CNN) is trained using the change-based features above and the stepped score (“step up”, “step down” or “no change”) as target.

In phase 2, the model trained in the fourth step P1S4 of phase 1 is applied to real-time data obtained from user 24 in order to detect a potential change in severity of symptoms for user 24.

In the first step P2S1 of phase 2, data is collected that reflects the mental state of user 24, e.g., physiological data obtained from passive sensors. This first step P2S1 of phase 2 is thus similar to the data collection step P1S1 of phase 1, in which data from multiple individuals is collected and stored in the database. However, in the first step P2S1 of phase 2, the data from the questionnaires is collected only once at the start of the interaction of user 24 with the system to establish a mental state as a starting point. For the rest of the period during which user 24 uses the system, only data acquired by sensors that monitor the user's physiological state without requiring the user to actively make statements are used.

In the second step P2S2 of phase 2, the same features computed at phase 1 are created on the new data collected during this phase. The set of features created and the length of time period preferably match those used in phase 1.

In the third step P2S3 of phase 2, the model trained in phase 1 is used to detect changes in the mental state of user 24 based on the acquired data. The features created in the previous step serve as input to the model, and the state change over the time period is classified into step-up, step-down or no change.

In the first step P3S1 of phase 3, the system decides based on the detected state change whether the individual is to be presented with a milder or a more intensive content, or whether the same content should continued to be displayed.

Based on the detection, a triage system of phase 3 is employed for delivering tailored interventions. If a step up in severity is detected, user 24 can be referred to a different content based on the updated severity of symptoms. In case of digital mental healthcare, if a step-up change is detected, user 24 can be referred to specific digital content, such as a Cognitive Behavioral Therapy digitally provided via the mobile app.

If a step down in severity is detected, the user can be referred to a different, milder content. In case of digital mental healthcare, user 24 can be referred to a preventative or a wellness app, such as the mobile app providing mindfulness, sleep programs, assistance in building healthy habits, cognitive restructuring for self-esteem, low mood, worry etc.

If no change is detected, in case of no or mild symptoms, user 24 would continue to be presented with the same content, whereas if the ongoing content did not result in an expected improvement, a clinician could be informed.

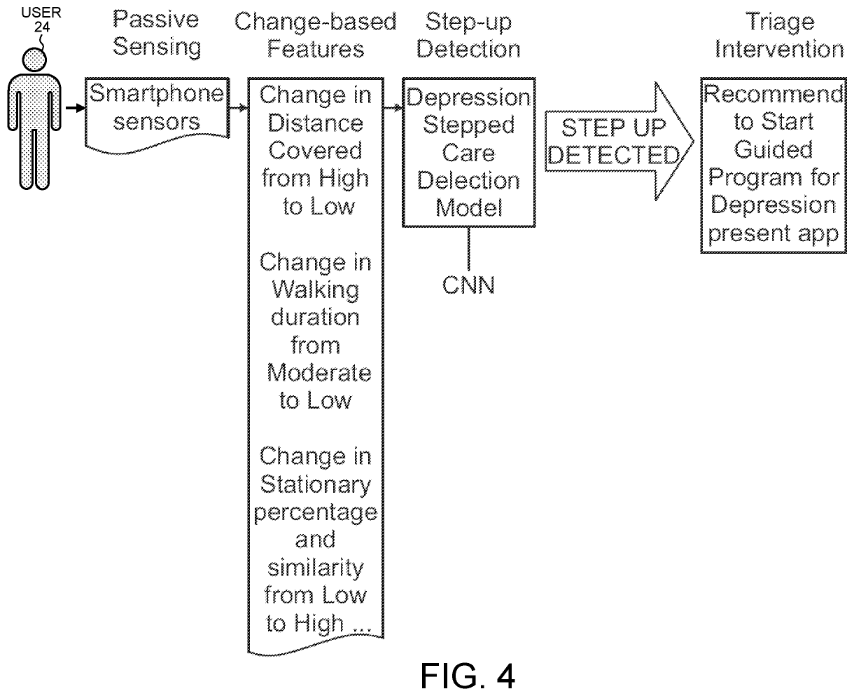

FIG. 4 is a flowchart of the operation of the stepped mental health care system as applied to the individual user 24. User 24 is using the mobile app on smartphone 10 to manage stress at a sub-clinical level. The system collects data reflecting the user's physiological state and behavior using smartphone sensors, such as a gyrometer, a camera, etc. The data is acquired passively and/or continuously without user 24 having to take any specific action.

In this example, the model detects a step-up in the severity of symptoms for user 24 based on the change-based features computed between Week 0 and Week 2 and recommends that user 24 start a guided program for depression. The content displayed to user 24 is thus a guided program for depression delivered through the mobile app. The data collection and features creation for this example are described in more detail below.

Initially, data are collected and aggregated, examples of which are given below. Based on the acquired smartphone sensors data, the system aggregates the data into the following smartphone sensors and event logs:
data_accumulatedSteps: the number of steps during a segment;
data_averageActivePace: average pace at which user 24 moved;
data_currentCadence: The rate at which steps are taken, measured in steps per second;
data_currentPace: the last pace of user 24, measured in seconds per meter;
data_distance: total distance covered, provided by CoreMotion on iOS or calculated internally on Android in order to provide an estimation;
data_duration: number of seconds the segment covered;
data_floorsAscended: the number of floors ascended by the user during a segment;
data_floorsDescended: the number of floors descended by the user during a segment;
data_activityCountFor24h: the total number of events detected;
data_longestInterruptedRest: a feature allowing for a brief gap to no count as an interruption of the rest;
data_longestStationary: the longest period of lack of phone physical movement or the longest period screen is turned off.

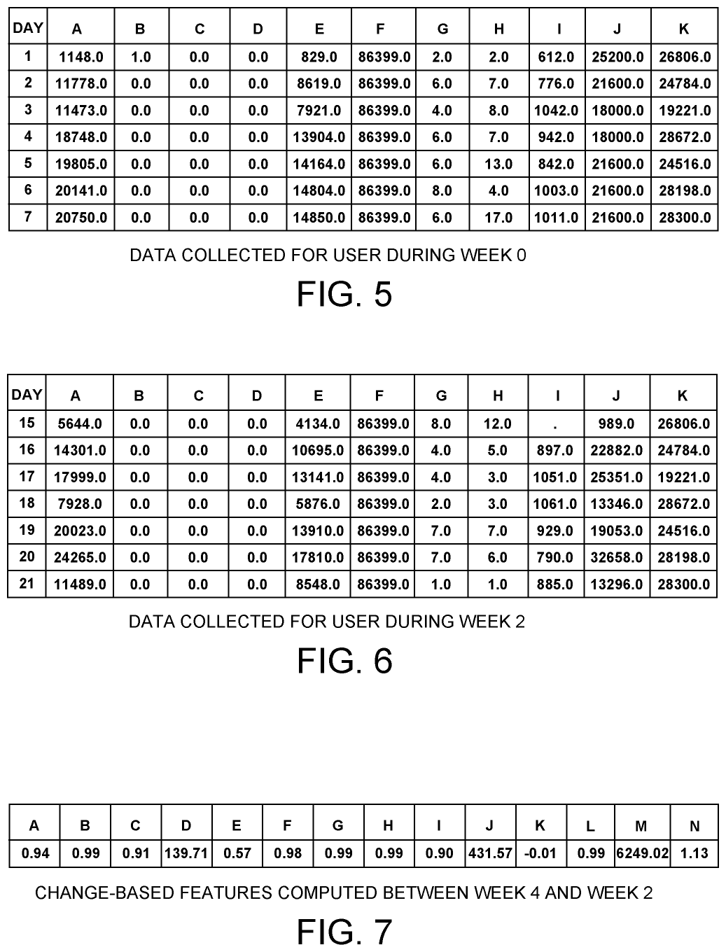

FIGS. 5-6 are tables that illustrates numerical examples of data collected for user 24 during week 0 and week 2, respectively. The tables illustrate the raw data collected using the sensor library. The features shown in FIGS. 5-6 and labeled as A through K are merely illustrative and not limiting. The features labeled A-K correspond to the following:
A=data_accumulatedStep
B=data_averageActivePace
C=data_currentCadence
D=data_currentPage
E=data_distance
F=data_duration
G=data_floorsAscended
H=data_floorsDescended
I=data_activityCountFor24h
J=data_longestInterruptedRest
K=data_longestStationary

Ground-truth information about the change in the conscious mental health state of user 24 was captured by the PHQ-8 questionnaire, reported by individuals/users three times: baseline, at the 2-week point, and at the 4-week point.

The state change is defined as “step up” if the individual has a level of severity in the second questionnaire result that is higher than that of the first. The state change is defined as “step down” if the level of severity in the second questionnaire is lower than that of the first. And the state change is defined as “no-change” if both reports indicate the same severity range. For PHQ-8, the severity of depression symptoms is classified based on a score arising from the questionnaire that is classified according to the following classes: 0-4=“none”, 5-9=“mild”, 10-14=“moderate”, 15-24=“severe”.

In this example, user 24 answers a PHQ-8 questionnaire at onboarding and scores 4, classified as “None”. The user answers the same questionnaire at week 2 and week 4, scoring 8 and 10 respectively, corresponding to initially mild but then moderate depression.

In this particular case, the CNN model identifies indications of the mental state of user 24 deteriorating corresponding to a “step up” in the content that should be presented to user 24 because the user stepped up in symptom severity.

In this particular example, the following aggregation methods for the features were used in the first step P1S1 of phase 1:
Absolute change in mean between weeks 0 and 2 (or 2 and 4);
Absolute change in mean between week 0, week 1 and week 2 (or 2, 3 and 4);
Relative change in mean/standard deviation between weeks 0 and 2 (or 2 and 4);
Relative change in mean/standard deviation between week 0, week 1 and week 2 (or 2, 3 and 4)
Normalized regression slope over 2 weeks
Cosine similarity between weeks 0 and 2 (or 2 and 4) for subsample of:
   top 3 daily values;
   bottom 3 daily values.

This generated about 100 features (# sensors×# aggregation methods). Thereafter, the correlations between features versus targets are determined, and 14 features with statistically significant correlation to the targets (“step up”, “step down”, “no change”) were indentified to be used for predictive modeling. The features used in this example are:
data_distance_similarity_low_vals: cosine similarity between weeks 0 and 2 of the bottom 3 daily values of distance;
data_daytimeActivity_similarity_low_vals: cosine similarity between weeks 0 and 2 of the bottom 3 daily values of daytime activity;
distance_covered_wk_mean_change: relative change in mean between weeks 0 and 2 of the total distance daily;
data_distance_mean_diff: absolute change in mean between weeks 0 and 2 of the total distance daily;
data_floorsAscended_mean_diff: absolute change in mean between weeks 0 and 2 of the floors ascended daily;
data_stepSize_wk_mean_change: relative change in mean between weeks 0 and 2 of the step size;
inefficiency_similarity_low_vals: cosine similarity between weeks 0 and 2 of the bottom 3 daily values of inefficiency of swiping movements;
data_longestStationary_similarity: cosine similarity between weeks 0 and 2 of daily values of longest time stationary between 5 pm and next 5 pm;
data_longestStationary_similarity_low_vals: cosine similarity between weeks 0 and 2 of the bottom 3 daily values of longest time stationary between 5 pm and next 5 pm;
mean_walking_duration_diff_wk: absolute change in mean between weeks 0 and 2 of the total walking duration daily;
mean_stationary_perc_diff_wk: absolute change in mean between weeks 0 and 2 of the percentage walking duration daily;
ratio_stationary_perc_diff_wk: ratio between the mean in weeks 0 and 2 of the percentage time stationary;
mean_longesstationary_stationary_diff_wk: absolute change in mean between weeks 0 and 2 of the longest time stationary between 5 pm and next 5 pm;
ratio_longesstationary_stationary_diff_wk: ratio between the mean in weeks 0 and 2 of the longest time stationary between 5 pm and next 5 pm;

FIG. 7 is a table that illustrates change-based features computed for user 24. In this example, cosine similarity was used to determine similarity between feature vectors. A value of 1.00 indicates that there was no change. The similarity indicated in the table of FIG. 7 compares the features indicated in the columns of FIGS. 5-6. The change-based features of FIG. 7 are:
A=data_distance_similarity_low_vals;
B=data_daytimeActivity_similarity_low_vals;
C=distance_covered_wk_mean_change;
D=data_distance_mean_diff;
E=data_floorsAscended_mean_diff;
F=data_stepSize_wk_mean_change;
G=inefficiency_similarity_low_vals;
H=data_longestStationary_similarity;
I=data_longestStationary_similarity_low_vals;
J=mean_walking_duration_diff_wk;
K=mean_stationary_perc_diff_wk;
L=ratio_stationary_perc_diff_wk;
M=mean_longesstationary_stationary_diff_wk;
N=ratio_longesstationary_stationary_diff_wk.

As can be seen in the table of FIG. 7, changes were detected in fields D, J an M corresponding to changes in the distance covered by user 24, the walking duration and the stationary time of user 24. In summary, user 24 changed to a more sedentary life style over the course of the four weeks that have been investigated.

Data regarding these fourteen change features A-N and the questionnaire data (as ground truth data) were then used to train a convolutional neural network to predict changes in the mental state of user 24 based on detected feature changes.

As an example of a machine learning model or CNN, XGBoost can be used. In this example, the fourteen features A-N were used with data collected from all users as an input for the XGBoost model and with the step-up depression score as a target.

XGBoost represents an optimized implementation of gradient boosted decision trees, although any other machine learning algorithm or CNN can be employed for this task.

After training, the CNN detects based on the detected feature changes shown in FIG. 7 that user 24 is highly likely to be experiencing a step up in the user's symptoms, and the triage system recommends that the user start a guided program for depression. Suitable content, e.g., an app guiding user 24 through a self-help program, can be displayed on the user's smartphone 10. The identified increase in symptom severity detected by the CNN was confirmed by the questionnaire results of user 24.

Although the present invention has been described in connection with certain specific embodiments for instructional purposes, the present invention is not limited thereto. Accordingly, various modifications, adaptations, and combinations of various features of the described embodiments can be practiced without departing from the scope of the invention as set forth in the claims.

What is claimed is:

1. A system for determining a mental health state of a user and for adjusting output content accordingly, the system comprising:
a monitoring unit configured to monitor parameters of the user and to acquire corresponding data;
an analysis unit configured to extract features from data acquired by the monitoring unit;
a classification unit configured to detect a change in the mental health state of the user based on the features extracted by the analysis unit and to classify the change in the mental health state of the user;
a control unit configured to adapt content that is output to the user by an output unit based on the change of the mental health state of the user detected by the classification unit; and
the output unit configured to output the content to the user based on the change in the mental health state of the user.

2. The system of claim 1, further comprising:
a prediction unit configured to predict the change in the mental health state of the user in the future based on the features extracted by the analysis unit and based on changes in the features extracted by the analysis unit.

3. The system of claim 1, wherein the monitoring unit is configured to acquire data selected from the group consisting of: physiological data of the user, data derived from contextual input from the user such as ecological momentary assessment data, data from electronic health records, data reflecting behavior of the user, and data indicative of the mental health state of the user.

4. The system of claim 1, wherein the classification unit is configured to determine changes over time in features extracted from data acquired by the monitoring unit and to determine a change in the mental health state of the user based on changes in the features.

5. The system of claim 1, wherein the monitoring unit is configured to receive data indicative of a conscious mental health state of the user and to monitor physiological parameters of the user using a sensor, and wherein the system further comprises an integration unit configured to integrate the data indicative of the conscious mental health state of the user with the data regarding physiological parameters of the user indicative of a subconscious mental health state of the user.

6. The system of claim 2, wherein the prediction unit is configured to predict a change in the mental health state of the user in the future based on changes in the features detected by the analysis unit and data reflecting a conscious mental health state of the user acquired by the monitoring unit.

7. The system of claim 1, wherein the control unit is configured to adapt the content output to the user by the output unit based on a stepped care paradigm using a classification result of the classification unit, and wherein in the stepped care paradigm classes of mental states are associated with recommended content.

8. The system of claim 1, wherein the system is configured to determine a change in the mental health state of the user at a predetermined time interval, wherein the time interval has a length that is determined by the control unit based on a classification result of the classification unit.

9. The system of claim 1, wherein the output unit, the monitoring unit, the analysis unit, the classification unit and the prediction unit are part of a computing system on a smartphone.

10. The system of claim 1, further comprising:
an alert unit configured to output an alert via a device separate from the system if a classification result output by the classification unit falls within a predetermined class associated with a mental health condition that has a severity above a defined severity threshold.

11. The system of claim 10, wherein the alert unit is configured to output the alert if there has been no detectable change in the classification result for a predetermined time interval.

12. A method for determining a mental health state of a user and for adjusting output content accordingly, the method comprising:
monitoring parameters of the user and acquiring corresponding data using a monitoring unit;
extracting features from the data acquired by the monitoring unit using an analysis unit;
detecting a change in a mental health state of the user based on the features using a classification unit;
classifying the detected change in the mental state of the user into predetermined classes using the classification unit;
adapting content to be presented to the user based on the detected change in mental health state of the user, wherein the content is adapted using a control unit; and
presenting the content to the user using an output unit based on the detected change of the mental health state of the user.

13. The method of claim 12, wherein the monitoring unit, the analysis unit, the classification unit, the control unit and the output unit are part of a computing system on a smartphone.

14. The method of claim 12, wherein the data acquired in the monitoring step is selected from the group consisting of: physiological data of the user, data obtained from contextual input from the user, data from electronic health records, data reflecting a behavior of the user, and data indicative of the mental health state of the user.

15. The method of claim 12, further comprising:
determining changes over time in the features extracted from the data by the analysis unit; and
determining how the mental health state of the user changes based on the changes in the features.

16. The method of claim 12, further comprising:
predicting how the mental health state of the user will change in the future based on the features extracted from the data by the analysis unit and based on changes over time in the features extracted from the data by the analysis unit.

17. The method of claim 16, wherein the predicting how the mental health state of the user will change in the future is based on data indicative of a conscious mental state of the user acquired by the monitoring unit.

18. The method of claim 12, further comprising:
acquiring data indicative of a conscious mental state of the user;
acquiring physiological parameters of the user indicative of a subconscious mental state of the user using a sensor; and
synchronizing the data indicative of the conscious mental state of the user with the physiological parameters indicative of the subconscious mental state of the user using an integration unit.

19. The method of claim 12, wherein the content to be presented to the user is adapted by classifying mental health states into classes that are associated with types of content to be presented to the user.

20. The method of claim 12, wherein the method is performed at predetermined time intervals to determine the mental health state of the user, and wherein the time intervals have lengths determined by the control unit by classifying mental health states into classes that are associated with types of content to be presented to the user.

21. The method of claim 12, further comprising:
outputting an alert to the user if a classification result output by the classification unit falls within a predetermined class associated with a mental health condition that has a severity above a predefined severity threshold.
